(19) United States
(12) Patent Application Publication
(10) Pub. No.: US 2010/0313888 A1
(43) Pub. Date: Dec. 16, 2010

(54) SPOOL VALVE ASSEMBLY FOR DELIVERY OF A GASEOUS DRUG

(75) Inventors: Richard Joseph Kocinski, North Huntington, PA (US); David Tomas, North Huntington, PA (US)

Correspondence Address: PEPPER HAMILTON LLP, ONE MELLON CENTER, 50TH FLOOR, 500 GRANT STREET, PITTSBURGH, PA, 15219, US

(73) Assignee: INSPIRED TECHNOLOGIES, INC., North Huntington, PA (US)

(21) Appl. No.: 12/485,734

(22) Filed: Jun. 16, 2009

(51) Int. Cl. A61M 16/20

(52) U.S. Cl. 128/204.23; 128/205.24

(57) ABSTRACT

A spool valve assembly for a gaseous drug delivery device includes a housing, a gas inlet, a continuous flow orifice, a valve outlet, a valve mounting surface, a spool valve, a patient outlet, an exhaust chamber with a pressure sensor detecting inhalation and a gaseous drug path to the patient, and a control circuit connected to the pressure sensor. The gas inlet includes a port connected to a gaseous drug supply, with a first portion inside the housing and a second portion outside the housing. The valve mounting surface includes a mount for a high flow valve and a low flow valve, with the valve mounting surface between the gas inlet and the valve outlet. The spool valve includes a knob, an outer surface, sealing members and a counter bore, with a first portion of the knob inside the housing and a second portion outside the housing.

### F. BACKGROUND

Ambulatory oxygen dosing systems are often used to treat patients with a respiratory disorder. The respiratory disorder, for instance, may be a lung disease such as chronic obstructive pulmonary disease (“COPD”). Prior art devices for oxygen delivery are typically non-feedback devices that deliver oxygen at a fixed flow and are known as conservers. Such devices involve a fixed flow at a variable time. For example, prior devices operate as on/off valves where the fixed flow is provided on demand. These valves turn on during inhalation and off during exhalation and supply a prescribed flow to a patient. The patient is restricted to the prescribed flow. Both of these devices do not adjust dosage with high breath rates that may occur as the patient ambulates, which may cause the patient to have low oxygen levels in the patient's bloodstream, or rather, desaturate. Through these devices, oxygen is wasted and not as efficiently or easily delivered to an ambulating patient or a patient at rest. Alternatively, an ambulating patient may not receive all of the oxygen that the patient needs at a particular point in time.

### G. SUMMARY

Before the present systems, devices and methods are described, it is to be understood that this disclosure is not limited to the particular systems, devices and methods described, as these may vary. It is also to be understood that the terminology used in the description is for the purpose of describing the particular versions or embodiments only, and is not intended to limit the scope.

It must also be noted that as used herein and in the appended claims, the singular forms “a,” “an,” and “the” include plural references unless the context clearly dictates otherwise. Unless defined otherwise, all technical and scientific terms used herein have the same meanings as commonly understood by one of ordinary skill in the art. Although any methods, materials, and devices similar or equivalent to those described herein can be used in the practice or testing of embodiments, the preferred methods, materials, and devices are now described. All publications mentioned herein are incorporated by reference. Nothing herein is to be construed as an admission that the embodiments described herein are not entitled to antedate such disclosure by virtue of prior invention. As used herein, the term “comprising” means “including, but not limited to.”

In an embodiment, a spool valve assembly for a device which delivers a gaseous drug to a patient with a respiratory disorder may include a housing, a gas inlet, a continuous flow orifice, a valve outlet, a valve mounting surface, a spool valve, a patient outlet, an exhaust chamber, through which a pressure sensor detects patient inhalation and the gaseous drug passes to arrive at the patient outlet, and a control circuit connected to the pressure sensor. The gas inlet may include a gas inlet port for connection to a gaseous drug supply, wherein at least a first portion of the gas inlet port is positioned inside the housing and a second portion is positioned outside the housing. The valve mounting surface may include a mount for at least a high flow valve and a low flow valve, wherein the valve mounting surface is positioned between the gas inlet and the valve outlet. The spool valve may include a knob, an outer surface, one or more sealing members and a counter bore, wherein at least a first portion of the knob is positioned inside the housing and a second portion is positioned outside the housing.

In an embodiment, a dosing apparatus for providing a dose of a gaseous drug to a patient with a respiratory disorder may include a gaseous drug delivery device allowing a variation of a volume of the gaseous drug delivered to the patient, and a power supply that provides power to the apparatus. The gaseous drug delivery device may include a spool valve assembly. The spool valve assembly may include a housing, a gas inlet, a continuous flow orifice, a valve outlet, a valve mounting surface, a spool valve, a patient outlet, and an exhaust chamber. The gas inlet may include a gas inlet port for connection to a gaseous drug supply, wherein at least a first portion of the gas inlet port is positioned inside the housing and a second portion is positioned outside the housing. The valve mounting surface may include a mount for at least a high flow valve and a low flow valve, wherein the valve mounting surface is positioned between the gas inlet and the valve outlet. The spool valve may include a knob, an outer surface, one or more sealing members and a counter bore, wherein at least a first portion of the knob is positioned inside the housing and a second portion is positioned outside the housing. The exhaust chamber may include a pressure sensor port connecting the patient outlet to a pressure sensor to detect inhalation and transmit patient inhalation information to a control circuit, wherein the control circuit is connected to the pressure sensor.

### H. BRIEF DESCRIPTION OF THE DRAWINGS

Aspects, features, benefits and advantages of the present application will be apparent with regard to the following description and accompanying drawings, of which:

FIG. 1 depicts a three-dimensional view of an exemplary spool valve assembly for a gaseous drug delivery device under negative pressure, which delivers a gaseous drug to a patient with a respiratory disorder according to an embodiment.

FIG. 2 illustrates a top view of the spool valve assembly for a gaseous drug delivery device under negative pressure, which delivers a gaseous drug to a patient with a respiratory disorder according to an embodiment.

FIG. 3 illustrates a side view of the spool valve assembly for a gaseous drug delivery device under negative pressure, which delivers a gaseous drug to a patient with a respiratory disorder according to an embodiment.

FIG. 4 depicts a three-dimensional view of an exemplary spool valve assembly for a gaseous drug delivery device under positive pressure and in a continuous flow position, which delivers a gaseous drug to a patient with a respiratory disorder according to an embodiment.

FIG. 5 depicts a three-dimensional view of an exemplary spool valve assembly for a gaseous drug delivery device under negative pressure, which delivers a gaseous drug to a patient with a respiratory disorder according to another embodiment.

FIG. 6 illustrates a method for delivering a dose of a gaseous drug to a patient with a respiratory disorder through use of a spool valve assembly with a drug delivery device according to an embodiment.

FIG. 7 illustrates a method for providing a dose of a gaseous drug to an ambulating patient who has a respiratory disorder through use of a spool valve assembly with a drug delivery device according to an embodiment.

### I. DETAILED DESCRIPTION

For the purposes of the discussion below, a “valve” is a device that regulates the flow of a fluid. The fluid may include gases, fluidized solids, slurries, liquids and/or the like. The device may regulate the flow of fluid by opening, closing, or partially obstructing various passageways.

For the purposes of the discussion below, a “port” is an aperture through which a gas or fluid may flow and which typically is adapted for being physically connected to some other device. The port may be connected to the other device with threaded fitting, a socket and plug, and/or the like.

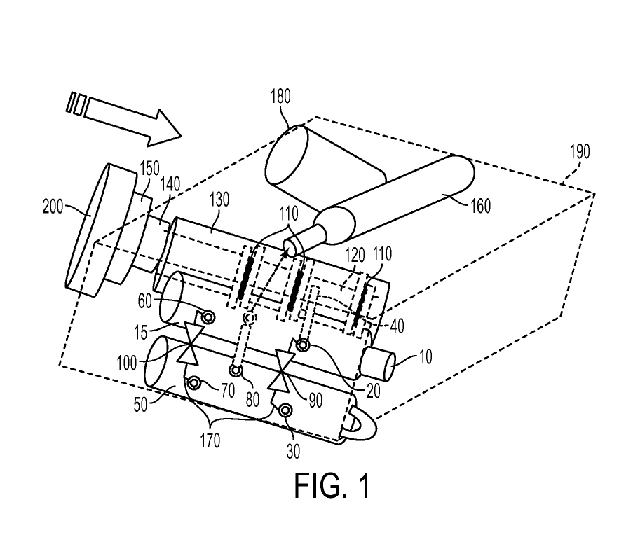

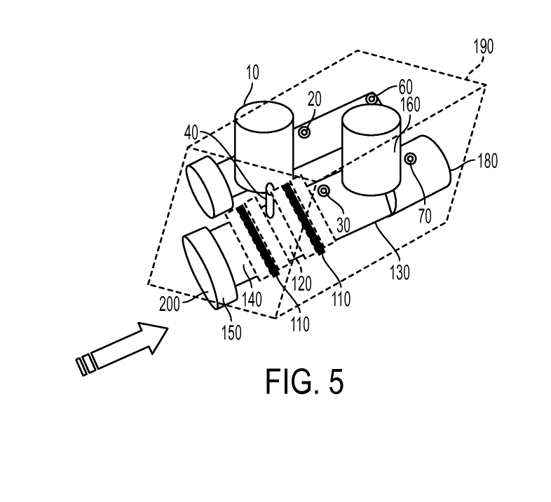

FIGS. 1-5 depict views of exemplary spool valve assemblies for a device which delivers a gaseous drug to a patient, such as a patient with a respiratory disorder. FIGS. 1-4 depict views of an exemplary spool valve assembly according to one embodiment, and FIG. 5 depicts a view of an exemplary spool valve assembly according to another embodiment. A dosing apparatus may include a gaseous drug delivery device and a power supply to power the apparatus. An exhaust chamber in the dosing apparatus may include a pressure sensor port connecting a patient outlet to a pressure sensor to detect inhalation and transmit patient inhalation information to a control circuit, wherein the control circuit may be connected to the pressure sensor. The gaseous drug delivery device may allow for a variation of a volume of the gaseous drug delivered to the patient. For example, the gaseous drug delivery device may be of the type disclosed in U.S. patent application Ser. No. 11/684,370, entitled “A Method and Apparatus for Delivering a Dose of a Gaseous Drug to a Patient,” the disclosure of which is incorporated herein by reference in its entirety.

As shown in FIG. 1, the assembly may include a housing 190. The housing 190 may include a gas inlet 15. The gas inlet 15 may include a gas inlet port 10 for connection to a gaseous drug supply. In an embodiment, the gaseous drug supply may be a gaseous or liquid oxygen received from a liquid oxygen reservoir and/or the like. Additional and/or alternative gaseous drug supplies may be utilized within the scope of this disclosure. At least a first portion of the gas inlet port 10 may be positioned inside the housing 190. Optionally, a second portion may be positioned outside or on the housing 190. In an embodiment, the gas inlet 15 may include a first valve inlet port 20 that directs flow to a high flow valve 90 and a second valve inlet port 60 that directs flow to a low flow valve 100.

The housing may include a continuous flow orifice 40. In an embodiment, the continuous flow orifice 40 may be provided through the gas inlet port 10. Alternatively, the continuous flow orifice 40 may be provided through the first valve inlet port 20. For example, the continuous flow orifice 40 may be in fluid communication with the first valve inlet port 20.

The assembly may include a valve outlet 50. In an embodiment, the valve outlet 50 may include a first valve outlet port 30 and a second valve outlet port 70. The first valve inlet port 20 may be in fluid communication with the first valve outlet port 30 via the high flow valve 90. The second valve inlet port 60 may be in fluid communication with the second valve outlet port 70 via the low flow valve 100. In an embodiment, the valve outlet 50 may include a third valve outlet port 80 that is in fluid communication with the exhaust chamber 160. In an embodiment, the valve outlet 50 may be closed off and not threaded to receive a fitting. Alternatively, the valve outlet 50 may include a connection port 55 that is threaded to receive a fitting.

The housing 190 may include a valve mounting surface 170. The valve mounting surface 170 may include a mount for at least a high flow valve 90 and a low flow valve 100. The valve mounting surface 170 may be positioned between the gas inlet 15 and the valve outlet 50. For example, the high flow valve 90 may be mounted upon the valve mounting surface 170 and be in fluid communication with the first valve inlet port 20 and with the first valve outlet port 30. Additionally, the low flow valve 100 may be mounted upon the valve mounting surface 170 and be in fluid communication with the second valve inlet port 60 and with the second valve outlet port 70. In an embodiment, one or more of the high flow valve 90 and the low flow valve 100 may open upon a pressure sensor (not shown) detecting a negative pressure, or vacuum, within the exhaust chamber 160.

The housing 190 may include a spool valve 200. The spool valve 200 may include a knob 150, an outer surface 130, one or more sealing members 110 and a counter bore 140. At least a first portion of the knob 150 may be positioned inside the housing 190 and a second portion may be positioned outside the housing 190. In an embodiment, the outer surface 130 may be cylindrical, rectangular, and/or the like. In an embodiment, the sealing members 110 may be o-rings and/or the like. In an embodiment, the sealing members 110 may be arranged at least at three separate locations on the spool valve 200. The outer surface 130 of the spool valve 200 may have a diameter that is smaller than that of the one or more sealing members 110 to provide an effective seal around one or more gas delivery channels 120. Between the seals, an area between the one or more sealing members 110 and the outer surface 130 of the spool valve 200 may form the one or more gas delivery channels 120. In another embodiment, the sealing members 110 may be integral with the outer surface 130 of the spool valve 200 to form one piece.

In an embodiment, the spool valve 200 may be inserted into the housing 190 through a hole at an outer surface of the housing. The hole may add generous lead in for insertion of the spool valve 200 into the housing 190 so that the one or more sealing members 110 are compressed upon the insertion. The hole may be sized for slight engagement of the sealing members 110 to minimize friction but allow for an effective seal around the one or more gas delivery channels 120.

Also, the assembly may include a patient outlet 180 through which the gaseous drug is delivered to the patient, and through which a patient's inhalation and exhalation may pass. In an embodiment, the patient outlet 180 may be threaded to receive a cannula barb fitting. A pressure sensor port may connect the patient outlet 180 to a pressure sensor to detect inhalation, and the pressure sensor may be connected to a control circuit. The pressure sensor may detect positive and/or negative pressure within the exhaust chamber 160. Negative pressure may also be referred to as a vacuum. In an embodiment, one or more of the high flow valve 90 and the low flow valve 100 may open upon the pressure sensor detecting a negative pressure, or vacuum, within the exhaust chamber 160.

In an embodiment, as shown in FIG. 1 in a position that may also be referenced as a non-continuous flow position, the spool valve 200 may be in a first position so that at least one sealing member 110 seals the continuous flow orifice 40 from the exhaust chamber 160. Additionally, the valve outlet 50 may include a third valve outlet port 80, and the spool valve 200 may align the third valve outlet port 80 with an opening to the exhaust chamber 160. A gas delivery channel may be formed from the valve outlet port 80 to an opening of the exhaust chamber 160.

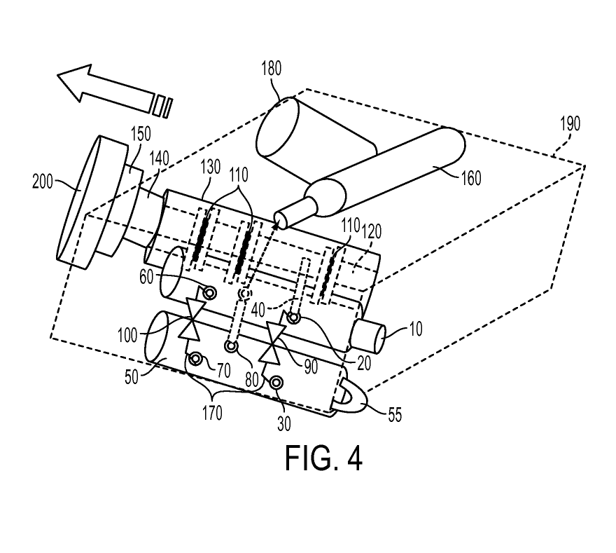

Alternatively, as shown in FIG. 4 in a position that may also be referenced as a continuous flow position, the spool valve 200 may be in a second position so that the spool valve 200 may form a gas delivery channel from the continuous flow orifice 40 to the exhaust chamber 160. In an embodiment, the pressure sensor may detect a positive pressure and prevent the opening of one or more of the high flow valve 90 and the low flow valve 100.

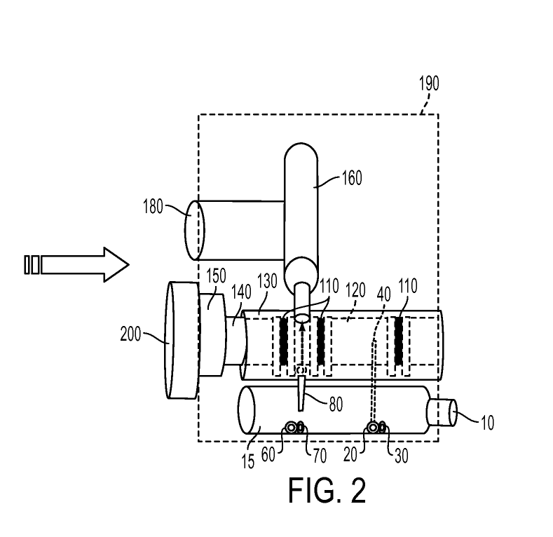

In an embodiment, the control circuit (not shown) may control the continuous flow orifice 40, the high flow valve 90 and/or the low flow valve 100, where the valves are shown in FIGS. 1 and 2. Additionally, a flow rate for the continuous flow orifice 40 may include a flow rate of 2 liters per minute. In another embodiment, a flow rate for the high flow valve 90 may include a flow rate of 9 liters per minute. In an embodiment, a flow rate for the high flow valve 90 may include a flow rate range of 8 to 10 liters per minute. A flow rate for the low flow valve 100 may include a flow rate of 4 liters per minute. In an embodiment, a flow rate for the low flow valve 100 may include a flow rate range of 3.5 to 4 liters per minute. In an embodiment, a combination of the high flow valve 90 and the low flow valve 100 may provide a flow rate range of 3 to 18 liters per minute. Optionally, the combination of the high flow valve 90 set to 8 liters per minute and the low flow valve 100 set to 4 liters per minute may provide a flow rate range of 10 to 11 liters per minute. Other flow rate ranges provided by a combination of the high flow valve 90 and the low flow valve 100 may be possible within the scope of this disclosure. In an embodiment, the assembly further may include mounting holes. The mounting holes may be used to mount the assembly to an object or to mount an object onto the assembly. In an embodiment, the object may be a printed circuit board.

FIGS. 1-3 reflect an embodiment illustrated in a first non-continuous flow position. A second continuous flow position is shown in FIG. 4.

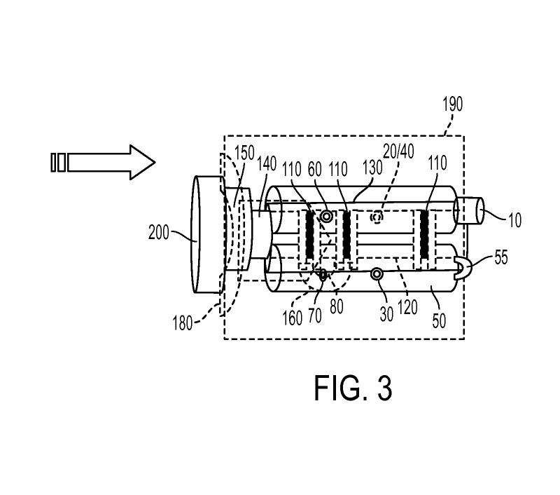

FIGS. 1-3 depict three-dimensional, top, and side views of an exemplary spool valve assembly for a gaseous drug delivery device under negative pressure and in a first non-continuous flow position according to an embodiment. FIGS. 1-3 depict a third valve outlet port 80 that may align with an opening to the exhaust chamber 160. FIG. 1 depicts a three-dimensional view of an exemplary spool valve assembly for a gaseous delivery device under negative pressure, which delivers a gaseous drug to a patient with a respiratory disorder according to an embodiment. FIG. 2 illustrates a top view of the spool valve assembly for a gaseous delivery device under negative pressure, which delivers a gaseous drug to a patient with a respiratory disorder according to an embodiment. FIG. 3 illustrates a side view of the spool valve assembly for a gaseous delivery device under negative pressure, which delivers a gaseous drug to a patient with a respiratory disorder according to an embodiment.

FIG. 4 depicts a three-dimensional view of an exemplary spool valve assembly for a gaseous delivery device under positive pressure and in a continuous flow position, which delivers a gaseous drug to a patient with a respiratory disorder according to an embodiment. FIGS. 1-4 illustrate a gas inlet port 10 that may be positioned on the side of the housing 190. FIGS. 1-4 also depict an exhaust chamber 160 that may be positioned on side facing the rear of the housing 190. In FIGS. 1-4, the continuous flow orifice may be provided through the first valve inlet port 20.

FIG. 5 reflects another embodiment in the first non-continuous flow position. FIG. 5 depicts a three-dimensional view of an exemplary spool valve assembly for a gaseous delivery device under negative pressure and in a non-continuous flow position, which delivers a gaseous drug to a patient with a respiratory disorder according to another embodiment. FIG. 5 illustrates a gas inlet port 10 that may be positioned on top of the housing 190. FIG. 5 also depicts an exhaust chamber 160 that may be positioned on top of the housing 190. In FIG. 5, the continuous flow orifice may be provided in a downwardly facing direction from the gas inlet port 10.

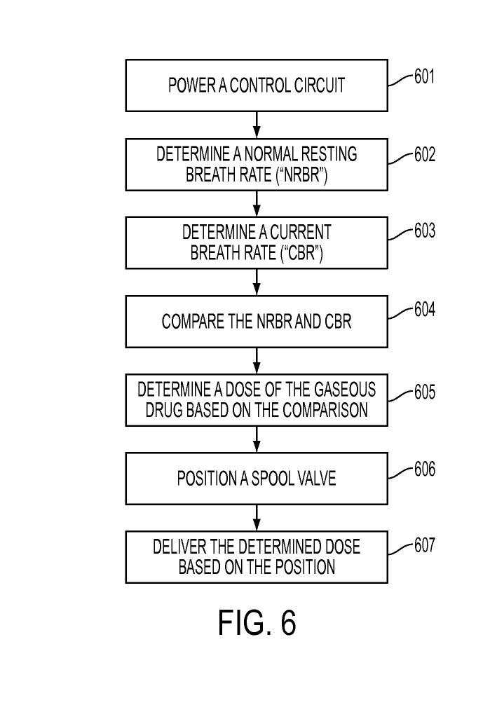

FIG. 6 illustrates a method for delivering a dose of a gaseous drug to a patient with a respiratory disorder through use of a spool valve assembly with a drug delivery device according to an embodiment. A control circuit may be powered 601 by a power supply. The control circuit may control a low flow valve, a high flow valve, and a continuous flow valve. A normal resting breath rate may be determined 602 for a patient through the control circuit. The control circuit may monitor a pressure sensor that changes its output upon patient inhalation. Through a signal change that may result from the pressure sensor output changes to indicate patient inhalation and exhalation transitions, the control circuit may determine a breath pattern including, but not limited to, length of breath, length of inhalation, length of exhalation, and a ratio of inhalation to exhalation.

A normal resting breath rate may be determined 602 by a calculation occurring during each breath of the patient. An algorithm may be utilized to smooth a previous normal resting breath rate against a current breath rate to determine a new normal resting breath rate. A current breath rate may be determined 603 for the patient through the control circuit. The current breath rate may be compared 604 to the normal resting breath rate. If the current breath rate exceeds a threshold, such as 3 breaths per minute over the normal resting breath rate, the algorithm may exclude the excessive breath and all subsequent breaths for an additional time period, such as two minutes. Determination 603 of the current breath rate may assist with calculating a delivery window for a next breath, which may be two-thirds of the inhalation of the current breath.

Based on the comparison 604, the dose of the gaseous drug for the patient may be determined 605. For example, a prescribed oxygen flow rate may be one of the following settings: 0, 0.5, 1.0, 2.0, 3.0, 4.0, and 5.0 standard liters per minute of oxygen, and a dose may be 16 cc of oxygen per each standard liters per minute of oxygen setting level. For instance, a flow selection of 1.0 standard liters per minute of oxygen may result in a dose of 16 cc. In an embodiment, if a patient's current breath rate exceeds 3 breaths per minutes over the normal resting breath rate, an additional dose of 16 cc may be delivered to a patient.

A spool valve within a spool valve assembly may be positioned 606. Based on the position 606 of the spool valve, the determined dose may be delivered 607. In an embodiment, the spool valve may be positioned 606 in an inward position so that a flow of the gaseous drug from a continuous flow valve is directed to a gas delivery channel that is sealed off from an exhaust chamber, wherein the exhaust chamber is in fluid communication with the patient. Positioning 606 the spool valve in an inward position may cause the device to deliver 607 the determined dose through the low flow valve, the high flow valve, or a combination of the low flow valve and the high flow valve. In another embodiment, a flow rate for the high flow valve may be 9 liters per minute. In an embodiment, a flow rate for the low flow valve may be 4 liters per minute. In an embodiment, the spool valve may be positioned 606 in an outward position so that a flow of the gaseous drug from a continuous flow valve is directed to a gas delivery channel in fluid communication with an exhaust chamber, wherein the exhaust chamber is in fluid communication with the patient. Positioning 606 the spool valve in an outward position may cause the device to deliver 607 the determined dose through the continuous flow valve. In an embodiment, a flow rate for the continuous flow valve may be 2 liters per minute.

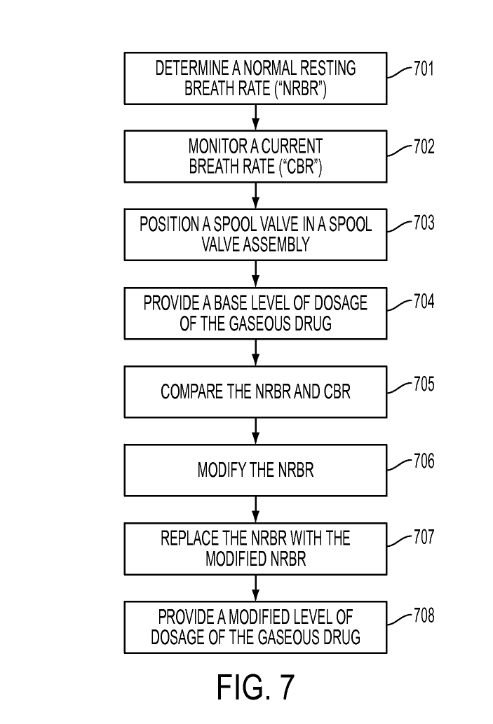

FIG. 7 illustrates a method for providing a dose of a gaseous drug to an ambulating patient who has a respiratory disorder through use of a spool valve assembly with a drug delivery device according to an embodiment. A normal resting breath rate for the patient may be determined 701 at a particular time through a control circuit. A current breath rate of the patient may be monitored 702. A spool valve within a spool valve assembly may be positioned 703. A base level of dosage of a gaseous drug may be provided 704 through the spool valve assembly. The current breath rate may be compared 705 to the normal resting breath rate by the control circuit. The normal resting breath rate may be modified 706 and determined via the control circuit based on the comparison 705 and the position 703 of the spool valve. The normal resting breath rate may be replaced 707 with the modified normal resting breath rate. A modified dosage level of the gaseous drug may be provided 708 through the spool valve assembly based on the modified normal resting breath rate. In an embodiment, the positioning 703 may include positioning the spool valve in an inward position, and providing the base level of dosage of the gaseous drug may include delivering the drug, as selected by the control circuit, through a low flow valve, a high flow valve, and/or a combination of the low flow valve and the high flow valve.

In an embodiment, if the comparison 705 reflects that the current breath rate is greater than or equal to three breaths per minute over the normal resting breath rate, providing 708 the modified dosage level may include providing of dosage of an additional sixteen cubic centimeters over the base level of dosage of the gaseous drug to the patient. Alternatively, if the comparison 705 reflects that the current breath rate is greater than or equal to six breaths per minute over the normal resting breath rate, providing 708 the modified dosage level may include providing of dosage of an additional thirty-two cubic centimeters over the base level of dosage of the gaseous drug to the patient.

In an embodiment, the method may further include locking the normal resting breath rate should the comparison 705 reflect that the current breath rate is greater than or equal to three breaths per minute over the normal resting breath rate. In embodiment, the method may further include unlocking the normal resting breath rate after a two minute delay once the comparison reflects that the current breath rate is less than three breaths per minute over the normal resting breath rate.

It will be appreciated that various of the above-disclosed and other features and functions, or alternatives thereof, may be desirably combined into many other different systems or applications. It will also be appreciated that various presently unforeseen or unanticipated alternatives, modifications, variations or improvements therein may be subsequently made by those skilled in the art which are also intended to be encompassed by the disclosed embodiments.

What is claimed is:

1. A spool valve assembly for a device which delivers a gaseous drug to a patient with a respiratory disorder, the assembly comprising:
a housing;
a gas inlet comprising a gas inlet port for connection to a gaseous drug supply, wherein at least a first portion of the gas inlet port is positioned inside the housing and a second portion is positioned outside the housing;
a continuous flow orifice;
a valve outlet;
a valve mounting surface comprising a mount for at least a high flow valve and a low flow valve, wherein the valve mounting surface is positioned between the gas inlet and the valve outlet;
a spool valve comprising a knob, an outer surface, one or more sealing members and a counter bore, wherein at least a first portion of the knob is positioned inside the housing and a second portion is positioned outside the housing;
a patient outlet;
an exhaust chamber, through which a pressure sensor detects patient inhalation and the gaseous drug passes to arrive at the patient outlet; and
a control circuit connected to the pressure sensor.

2. The assembly of claim 1,
wherein the gas inlet further comprises a first valve inlet port and a second valve inlet port,
wherein the valve outlet further comprises a first valve outlet port and a second valve outlet port, and
wherein the first valve inlet port is in fluid communication with the first valve outlet port and the second valve inlet port is in fluid communication with the second valve outlet port.

3. The assembly of claim 1, wherein one or more of the high flow valve and the low flow valve open upon the pressure sensor detecting a negative pressure.

4. The assembly of claim 1, wherein the continuous flow orifice is provided through the first valve inlet port.

5. The assembly of claim 1, wherein the continuous flow orifice is provided through the gas inlet port.

6. The assembly of claim 1, wherein when the spool valve is in a first position, at least one sealing member seals the continuous flow orifice from the exhaust chamber.

7. The assembly of claim 6, wherein the valve outlet comprises a third valve outlet port and the spool valve aligns the third valve outlet port with an opening to the exhaust chamber.

8. The assembly of claim 1, wherein when the spool valve is in a second position, the spool valve forms a channel from the continuous flow orifice to the exhaust chamber.

9. The assembly of claim 8, wherein the pressure sensor detects a positive pressure and prevents the opening of one or more of the high flow valve and the low flow valve.

10. The assembly of claim 1, wherein the one or more sealing members are arranged at least at three locations on the spool valve.

11. The assembly of claim 1, wherein the patient outlet is threaded to receive a cannula barb fitting.

12. The assembly of claim 1, wherein the valve outlet is closed off and not threaded to receive a barb fitting.

13. The assembly of claim 1, wherein the control circuit controls one or more of the following: the continuous flow orifice, the high flow valve and the low flow valve.

14. The assembly of claim 13, wherein a flow rate for the continuous flow orifice comprises a flow rate of 2 liters per minute.

15. The assembly of claim 13, wherein a flow rate for the high flow valve comprises a flow rate of 9 liters per minute.

16. The assembly of claim 13, wherein a flow rate for the low flow valve comprises a flow rate of 4 liters per minute.

17. The assembly of claim 1, further comprising one or more mounting holes.

18. The assembly of claim 1, wherein the gaseous drug supply is a liquid oxygen reservoir.

19. A dosing apparatus for providing a dose of a gaseous drug to a patient with a respiratory disorder, the apparatus comprising:
a gaseous drug delivery device allowing a variation of a volume of the gaseous drug delivered to the patient, the gaseous drug delivery device comprising a spool valve assembly, the spool valve assembly comprising:
a housing;
a gas inlet comprising a gas inlet port for connection to a gaseous drug supply, wherein at least a first portion of the gas inlet port is positioned inside the housing and a second portion is positioned outside the housing;
a continuous flow orifice;
a valve outlet;
a valve mounting surface comprising a mount for at least a high flow valve and a low flow valve, wherein the valve mounting surface is positioned between the gas inlet and the valve outlet;
a spool valve comprising a knob, an outer surface, one or more sealing members and a counter bore, wherein at least a first portion of the knob is positioned inside the housing and a second portion is positioned outside the housing;
a patient outlet; and
an exhaust chamber comprising a pressure sensor port connecting the patient outlet to a pressure sensor to detect inhalation and transmit patient inhalation information to a control circuit, wherein the control circuit is connected to the pressure sensor; and
a power supply that provides power to the apparatus.
